(19) United States
(12) Patent Application Publication
(10) Pub. No.: US 2015/0126708 A1
(43) Pub. Date: May 7, 2015

(54) RADIOFLUORINATION METHOD

(71) Applicants: GE HEALTHCARE LIMITED, LITTLE CHALFONT, BUCKINGHAMSHIRE (GB); IMPERIAL INNOVATIONS LIMITED, LONDON (GB)

(72) Inventors: Sajinder Kaur Luthra, Amersham (GB); Robin Fortt, London (GB); Ramala Osman Awais, Nottingham (GB); Eric Ofori Aboagye, London (GB); Graham Smith, Kingston-Upon-Hull (GB)

(73) Assignees: IMPERIAL INNOVATIONS LIMITED, LONDON (GB); GE HEALTHCARE LIMITED, LITTLE CHALFONT, BUCKINGHAMSHIRE (GB)

(21) Appl. No.: 14/366,449

(22) PCT Filed: Dec. 20, 2012
(86) PCT No.: PCT/EP2012/076274

§ 371 Date: Jun. 18, 2014

(30) Foreign Application Priority Data
Dec. 20, 2011 (GB) 1121911.0

(51) Int. Cl. C07B 59/00; A61K 51/04

(52) U.S. Cl. CPC C07B 59/002; A61K 51/0453
USPC 530/331; 548/255; 422/554

(57) ABSTRACT

The present invention provides a method of radio fluorination of biological targeting molecules (BTMs) with the radioisotope ¹⁸F. Also provided are novel conjugates useful in the ¹⁸F-radio fluorination method, and the use of such conjugates and automated synthesizer apparatus including cassettes for carrying out the method.

### FIELD OF THE INVENTION

The present invention provides a method of radiofluorination of biological targeting molecules (BTMs) with the radioisotope ¹⁸F. Also provided are novel conjugates useful in the ¹⁸F-radiofluorination method, and the use of such conjugates and automated synthesizer apparatus including cassettes for carrying out the method.

### BACKGROUND TO THE INVENTION

¹⁸F click-labelling of targeting peptides, giving products incorporating an ¹⁸F-fluoroalkyl-substituted triazole have been reported by Li et at [Bioconj. Chem., 18(6), 1987-1994 (2007)], and Hausner et at [J. Med. Chem., 51(19), 5901-5904 (2008)].

The applications of “click chemistry” in biomedical research, including radiochemistry, have been reviewed by Nwe et at [Cancer Biother. Radiopharm., 24(3), 289-302 (2009)]. As noted therein, the main interest has been in the PET radioisotope ¹⁸F (and to a lesser extent ¹¹C), plus “click to chelate” approaches for radiometals suitable for SPECT imaging such as ^{99m}Tc or ¹¹¹In. Glaser and Robins have reviewed the use of click chemistry in PET radiochemical labelling reactions, focusing on the radioisotopes ¹⁸F and ¹¹C [J. Lab. Comp. Radiopharm., 52, 407-414 (2009)].

WO 2006/067376 discloses a method for labelling a vector comprising reaction of a compound of formula (I) with a compound of formula (II):

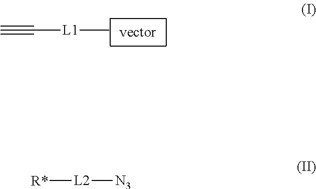

or, a compound of formula (III) with a compound of formula (IV):

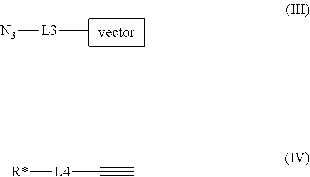

in the presence of a Cu(I) catalyst, to give a conjugate of formula (V) or (VI) respectively:

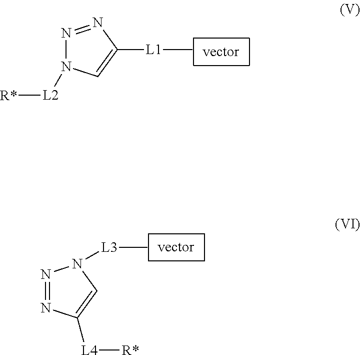

wherein:
L1, L2, L3, and L4 are each Linker groups;
R* is a reporter moiety which comprises a radionuclide.

R* of WO 2006/067376 is a reporter moiety which comprises a radionuclide, e.g. a positron-emitting radionuclide. Suitable positron-emitting radionuclides for this purpose are said to include ¹¹C, ¹⁸F, ⁷⁵Br, 76Br, ¹²⁴I, ⁸²Rb, ⁶⁸Ga, ⁶⁴Cu and ⁶²Cu, of which ¹¹C and ¹⁸F are preferred.

WO 2006/116629 (Siemens Medical Solutions USA, Inc.) discloses a method of preparation of a radiolabelled ligand or substrate having affinity for a target biomacromolecule, the method comprising:
(a) reacting a first compound comprising
   (i) a first molecular structure;
   (ii) a leaving group;
   (iii) a first functional group capable of participating in a click chemistry reaction; and optionally,
   (iv) a linker between the first functional group and the molecular structure, with a radioactive reagent under conditions sufficient to displace the leaving group with a radioactive component of the radioactive reagent to form a first radioactive compound;
(b) providing a second compound comprising
   (i) a second molecular structure;
   (ii) a second complementary functional group capable of participating in a click chemistry reaction with the first functional group, wherein the second compound optionally comprises a linker between the second compound and the second functional group;
(c) reacting the first functional group of the first radioactive compound with the complementary functional group of the second compound via a click chemistry reaction to form the radioactive ligand or substrate; and
(d) isolating the radioactive ligand or substrate.

WO 2006/116629 teaches that the method therein is suitable for use with the radioisotopes: ¹²⁴I, ¹⁸F, ¹¹C, ¹³N and ¹⁵O.

WO 2010/026388 teaches that compounds of formula:

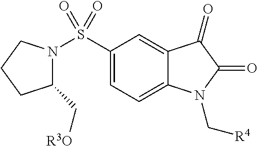

where:
R³ is phenyl, 3-fluorophenyl, 2,4-difluorophenyl, 3,5-difluorophenyl, an optionally substituted tetrahydropyran, an optionally substituted diazine or an optionally substituted triazole;
R⁴ is an optionally substituted phenyl or an optionally substituted triazole;
wherein when R is phenyl; R⁴ is an optionally substituted triazole;
   when labelled with ¹⁸F, are useful imaging agents.

A preferred compound of WO 2010/026388 is [¹⁸F]-ICMT-11:

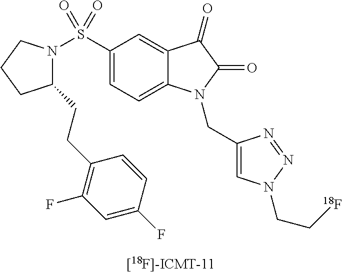 [SMILES: O=C1C(=O)N(Cc2cn(CC[18F])nn2)c2ccc(S(=O)(=O)N3CCC[C@H]3CCc3ccc(F)cc3F)cc21]

Smith et at [J. Med. Chem, 51(24), 8057-8067 (2008)] describe the synthesis of [¹⁸F]-ICMT-11 via click reaction using fluoroethylazide (¹⁸F—CH₂CH₂—N₃) from an alkyne-functionalised isatin precursor.

Glaser et at [Biorg. Med. Chem. Lett, 21, 6945-6949 (2011)] describe an improved radiosynthesis of [¹⁸F]-ICMT-11 using the acetal-protected alkyne-functionalised isatin precursor shown, and click reaction using fluoroethylazide:

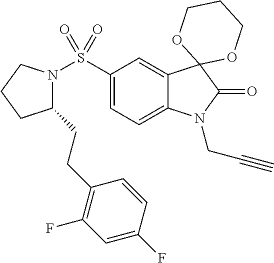 [SMILES: C#CCN1C(=O)C2(OCCCO2)c2cc(S(=O)(=O)N3CCC[C@H]3CCc3ccc(F)cc3F)ccc21]

The reactive dicarbonyl compound was protected to help suppress an unwanted impurity, suspected to be similar to [¹⁸F]-ICMT-11 and hence potentially a competing inhibitor for caspase-3 in vivo.

Smith et at [Poster 354 entitled “Fully Automated Synthesis of [¹⁸F]-ICMT-11 for Imaging Apoptosis”; 19^{th} International Symposium on Radiopharmaceutical Sciences, Amsterdam, 28 Aug. to 2 Sep. 2011; Abstract S443] describe an automated synthesis of [¹⁸F]-ICMT-11 via [¹⁸F]-fluoride displacement of the tosylate shown, followed by deprotection:

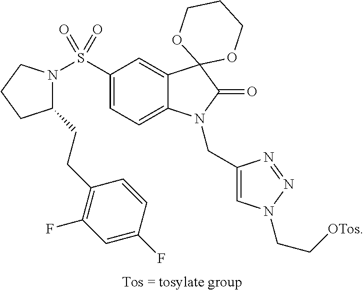

Smith et at do not describe how the tosylate is obtained.

There is therefore still a need for alternative radiofluorination methods, which provide radiofluorinated biological targeting molecules suitable for in vivo imaging. Ideally, the method needs to be suitable for automation, whereby radiopharmaceutical compositions can be obtained in a reproducible manner, in good radiochemical and chemical purity.

### The Present Invention.

The present invention provides alternative radiofluorination methods for the preparation of ¹⁸F-labelled triazole-functionalised biological targeting molecules.

The invention provides a simplified, more robust preparative methodology which is particularly useful for clinical applications. The radiofluorination method provides improved specific activity to maximize the signal from radiotracer/receptor interactions in vivo, and a reduction in the presence of potentially competitive stable impurities. The method is readily adaptable for automation. The method has the advantage that it uses [¹⁸F]-fluoride as the radioactive reactant—and thus avoids the need to prepare and handle volatile ¹⁸F-fluoroethylazide. That is beneficial because it minimizes the radiation dose to the operator by minimizing the synthesis steps involving radioactivity, and also minimizes the loss of radioactivity due to radioactive decay during synthesis elapsed time (¹⁸F has a half-life of 110 minutes).

In addition, the click reaction step is carried out non-radioactively, so the possible impurity issues arising from the copper used as a click catalyst Glaser et at [Biorg. Med. Chem. Lett, 21, 6945-6949 (2011)] can be resolved without the additional complication of radioactivity being present.

The present method facilitates radiosynthesis under Good Manufacturing Production (GMP) conditions, with an improved purity profile and increased radiochemical yield—thus allowing for multiple patient scans from a single preparation.

In the case of [¹⁸F]ICMT-11, the present inventors have established that the fluoroethylazide route provides a modest specific activity (1.2 GBq/μMol), with a stable isatin analogue impurity at a concentration of 14 μg/mL. The improved method of Glaser et at (cited above) provided [¹⁸F]ICMT-11 in a non-decay corrected end of synthesis (EOS) radiochemical yield of 3.0±2.6% (n=3), at a specific activity of 24±19 GBq/μmol and stable isatin impurity concentration of 4.1±4.1 μg/mL. The present method provides [¹⁸F]ICMT-11 in a radiochemical yield of 4.6±0.4 GBq (9.3±1.7% non-decay-correct radiochemical yield at EOS) in 90 minutes from target emptying to completion of aseptic dispensing. The radiochemical purity was 98-99% for all batches at end of synthesis, with a specific activity of 685±237 GBq/μmol. The total quantity of non-radioactive ICMT-11 and other impurities was shown to be 0.32±0.11 μg/mL and 1.06±0.24 μg/mL, respectively. No ICMT-11 precursor was detected. Those features together represent a significant improvement over prior art routes to [¹⁸F]ICMT-11.

### DETAILED DESCRIPTION OF THE PREFERRED ASPECTS OF THE INVENTION

In a first aspect, the present invention provides a method of ¹⁸F-radiofluorination of a biological targeting moiety, which comprises click reaction of a compound of Formula (I) with an azide of Formula (II):

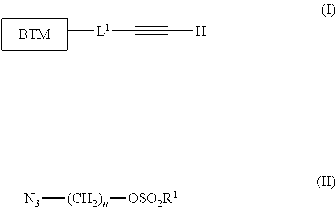

to give a conjugate of Formula (III):

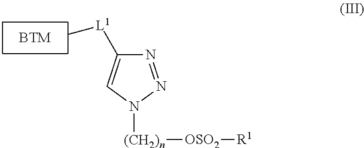

and reaction of the conjugate of Formula (III) with [¹⁸F]-fluoride to give the radiofluorinated product of Formula (IV):

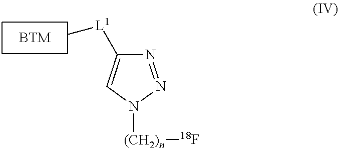

wherein:
L¹ is a linker group which may be present or absent;
n is 2, 3 or 4;
R¹ is C₁₋₄ alkyl; C₁ᵢ₋₄ fluoroalkyl; or —C₆H₄—R²,
   where R² is chosen from: H, CH₃, Br or NO₂;
BTM is the biological targeting moiety, optionally protected with one or more protecting groups.

The term “radiofluorination” has its conventional meaning, i.e. a radiolabelling process wherein the radioisotope used for the radiolabelling is a radioisotope of fluorine, here ¹⁸F.

When the linker group (L¹) is absent, that means that the alkyne group of Formula (I) is bonded directly to the BTM. That could mean for example, that the alkyne is conjugated to the side chain of an amino acid of a BTM peptide or protein, or directly to the N- or C-terminus of a BTM peptide. When present, each linker group (L¹) is preferably synthetic, and independently comprises a group of formula -(A)ₘ- wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block;

wherein each R is independently chosen from: H, C₁₋₄ alkyl, C₂₋₄ alkenyl, C₂₋₄ alkynyl, C₁₋₄ alkoxyalkyl or C₁₋₄ hydroxyalkyl;
and m is an integer of value 1 to 20.

By the term “biological targeting moiety” (BTM) is meant a compound which, after administration, is taken up selectively or localises at a particular site of the mammalian body in vivo. Such sites may for example be implicated in a particular disease state or be indicative of how an organ or metabolic process is functioning.

By the term “click reaction” has its conventional meaning, and here refers specifically to the reaction between an alkyne and an azide to give a triazole ring. Further details are given in J. Lahann (Ed), Click Chemistry for Biotechnology and Materials Science, Wily, (2009).

By the term “fluoroalkyl” is meant an alkyl group having at least one fluorine substituent up to an including a perfluoroalkyl group.

The term “protecting group” has its conventional meaning and refers to a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Suitable protecting groups are described in Protective Groups in Organic Synthesis, Theodora W. Greene and Peter G. M. Wuts, 4^{th} edition (John Wiley & Sons, 2007).

### Preferred Aspects.

When R¹ is fluoroalkyl, preferred such groups are chosen from: —CF₃ (triflate); —C₄F₉ (nonaflates) and —CH₂CF₃ (tresylates). R¹ is preferably chosen from —C₆H₄CH₃ (tosylate), —CH₃ (mesylate), C₆H₄NO₂ (nosylate) and —CF₃ and is most preferably tosylate.

The click reaction of is preferably carried out in the presence of a click catalyst. By the term “click catalyst” is meant a catalyst known to catalyse the click (alkyne plus azide) reaction. Suitable such catalysts are known in the art for use in click reactions. A preferred click catalyst comprises Cu(I). The Cu(I) catalyst is present in an amount sufficient for the reaction to progress, typically either in a catalytic amount or in excess, such as 0.02 to 1.5 molar equivalents relative to the azide of Formula (II). Suitable Cu(I) catalysts include Cu(I) salts such as CuI or [Cu(NCCH₃)₄][PF₆], but advantageously Cu(II) salts such as copper (II) sulfate may be used in the presence of a reducing agent to generate Cu(I) in situ. Suitable reducing agents include: ascorbic acid or a salt thereof for example sodium ascorbate, hydroquinone, metallic copper, glutathione, cysteine, Fe²⁺, or Co²⁺. Cu(I) is also intrinsically present on the surface of elemental copper particles, thus elemental copper, for example in the form of powder or granules may also be used as catalyst. Elemental copper, with a controlled particle size is a preferred source of the Cu(I) catalyst. A more preferred such catalyst is elemental copper as copper powder, having a particle size in the range 0.001 to 1 mm, preferably 0.1 mm to 0.7 mm, more preferably around 0.4 mm. Alternatively, coiled copper wire can be used with a diameter in the range of 0.01 to 1.0 mm, preferably 0.05 to 0.5 mm, and more preferably with a diameter of 0.1 mm. The Cu(I) catalyst may optionally be used in the presence of bathophenanthroline, which is used to stabilise Cu(I) in click chemistry.

Further details of suitable catalysts are described by Wu and Fokin [Aldrichim. Acta, 40(1), 7-17 (2007)] and Meldal and Tornoe [Chem. Rev., 108, 2952-3015 (2008)].

In the method of the first aspect, the compound of Formula (I) may optionally have one or more functional groups of the BTM protected with one or more protecting group(s)—to protect the BTM. Such protecting groups are as defined above. Typically, different protecting groups would be used for different functional groups. The method of the present invention tolerates a wide range of functional groups in the BTM. However, when the BTM comprises free thiol groups (e.g. a reduced cysteine-containing peptide), such thiol groups are preferably protected before the reaction of the first aspect is carried out. Similarly, any chelating functionalities or groups which coordinate well to copper(I) may require protection. Conditions for the introduction and removal of suitable protecting groups for different functional groups are described in the textbook by Greene et at (cited above). When such protecting group(s) are used, they are removed (ie. deprotected) after step (iii).

The BTM may be of synthetic or natural origin, but is preferably synthetic. The term “synthetic” has its conventional meaning, i.e. man-made as opposed to being isolated from natural sources eg. from the mammalian body. Such compounds have the advantage that their manufacture and impurity profile can be fully controlled. Monoclonal antibodies and fragments thereof of natural origin are therefore outside the scope of the term ‘synthetic’ as used herein. The BTM is preferably non-proteinaceous, i.e. does not comprise a protein.

The molecular weight of the BTM is preferably up to 10,000 Daltons. More preferably, the molecular weight is in the range 200 to 9,000 Daltons, most preferably 300 to 8,000 Daltons, with 400 to 6,000 Daltons being especially preferred. When the BTM is a non-peptide, the molecular weight of the BTM is preferably up to 3,000 Daltons, more preferably 200 to 2,500 Daltons, most preferably 300 to 2,000 Daltons, with 400 to 1,500 Daltons being especially preferred.

The biological targeting moiety preferably comprises: a 3-80 mer peptide, peptide analogue, peptoid or peptide mimetic which may be a linear or cyclic peptide or combination thereof; a single amino acid; an enzyme substrate, enzyme antagonist enzyme agonist (including partial agonist) or enzyme inhibitor; receptor-binding compound (including a receptor substrate, antagonist, agonist or substrate); oligonucleotides, or oligo-DNA or oligo-RNA fragments. More preferably, the BTM does not comprise a nucleoside or nitroimidzole.

The BTM is most preferably a 3-80 mer peptide or enzyme inhibitor.

By the term “peptide” is meant a compound comprising two or more amino acids, as defined below, linked by a peptide bond (i.e. an amide bond linking the amine of one amino acid to the carboxyl of another). The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids. The term “peptide analogue” refers to peptides comprising one or more amino acid analogues, as described below. See also Synthesis of Peptides and Peptidomimetics, M. Goodman et al, Houben-Weyl Vol E22c of ‘Methods in Organic Chemistry’, Thieme (2004).

By the term “amino acid” is meant an L- or D-amino acid, amino acid analogue (eg. naphthylalanine) or amino acid mimetic which may be naturally occurring or of purely synthetic origin, and may be optically pure, i.e. a single enantiomer and hence chiral, or a mixture of enantiomers. Conventional 3-letter or single letter abbreviations for amino acids are used herein. Preferably the amino acids of the present invention are optically pure. By the term “amino acid mimetic” is meant synthetic analogues of naturally occurring amino acids which are isosteres, i.e. have been designed to mimic the steric and electronic structure of the natural compound. Such isosteres are well known to those skilled in the art and include but are not limited to depsipeptides, retro-inverso peptides, thioamides, cycloalkanes or 1,5-disubstituted tetrazoles [see M. Goodman, Biopolymers, 24, 137, (1985)]. Radiolabelled amino acids such as tyrosine, histidine, methionine or proline are known to be useful in vivo imaging agents.

When the BTM is a peptide, it is preferably a 4-30 mer peptide, and most preferably a 5 to 28-mer peptide.

When the BTM is an enzyme substrate, enzyme antagonist, enzyme agonist, enzyme inhibitor or receptor-binding compound it is preferably a non-peptide, and more preferably is synthetic. By the term “non-peptide” is meant a compound which does not comprise any peptide bonds, i.e. an amide bond between two amino acid residues. Suitable enzyme substrates, antagonists, agonists or inhibitors include glucose and glucose analogues such as fluorodeoxyglucose; fatty acids, or elastase, Angiotensin II or metalloproteinase inhibitors. A preferred non-peptide Angiotensin II antagonist is Losartan. Suitable synthetic receptor-binding compounds include estradiol, estrogen, progestin, progesterone and other steroid hormones; ligands for the dopamine D-1 or D-2 receptor, or dopamine transporter such as tropanes; and ligands for the serotonin receptor.

When the BTM is an enzyme substrate, enzyme antagonist, enzyme agonist or enzyme inhibitor, preferred such biological targeting molecules of the present invention are synthetic, drug-like small molecules i.e. pharmaceutical molecules. Preferred dopamine transporter ligands such as tropanes; fatty acids; dopamine D-2 receptor ligands; benzamides; amphetamines; benzylguanidines, iomazenil, benzofuran (IBF) or hippuric acid.

When the BTM is a peptide, preferred such peptides include:
somatostatin, octreotide and analogues,
peptides which bind to the ST receptor, where ST refers to the heat-stable toxin produced by E. coli and other micro-organisms;
bombesin;
vasoactive intestinal peptide;
neurotensin;
laminin fragments eg. YIGSR, PDSGR, IKVAV, LRE and KCQAGTFALRGDPQG,
N-formyl chemotactic peptides for targeting sites of leucocyte accumulation,
Platelet factor 4 (PF4) and fragments thereof,
RGD (Arg-Gly-Asp)-containing peptides, which may eg. target angiogenesis [R. Pasqualini et al., Nat Biotechnol. 1997 June; 15(6):542-6]; [E. Ruoslahti, Kidney Int. 1997 May; 51(5):1413-7].
peptide fragments of α₂-antiplasmin, fibronectin or beta-casein, fibrinogen or thrombospondin. The amino acid sequences of α₂-antiplasmin, fibronectin, beta-casein, fibrinogen and thrombospondin can be found in the following references: α₂-antiplasmin precursor [M. Tone et al., J. Biochem, 102, 1033, (1987)]; beta-casein [L. Hansson et al, Gene, 139, 193, (1994)]; fibronectin [A. Gutman et al, FEBS Lett., 207, 145, (1996)]; thrombospondin-1 precursor [V. Dixit et al, Proc. Natl. Acad. Sci., USA, 83, 5449, (1986)]; R. F. Doolittle, Ann. Rev. Biochem., 53, 195, (1984);
peptides which are substrates or inhibitors of angiotensin, such as:
   angiotensin II Asp-Arg-Val-Tyr-Ile-His-Pro-Phe (E. C. Jorgensen et al, J. Med. Chem., 1979, Vol 22, 9, 1038-1044)
   [Sar, Ile] Angiotensin II: Sar-Arg-Val-Tyr-Ile-His-Pro-Ile (R. K. Turker et al., Science, 1972, 177, 1203).
Angiotensin I: Asp-Arg-Val-Tyr-Ile-His-Pro-Phe-His-Leu.

Preferred BTM peptides are RGD peptides. A more preferred such RGD peptide comprises the fragment:

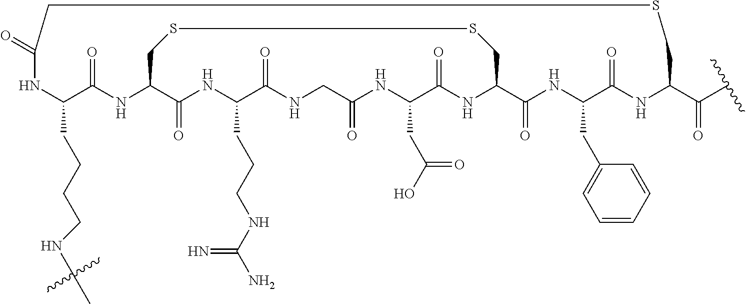 [SMILES: *NCCCC[C@@H]1NC(=O)CSC[C@@H](C(*)=O)NC(=O)[C@H](Cc2ccccc2)NC(=O)[C@@H]2CSSC[C@H](NC1=O)C(=O)N[C@@H](CCCNC(=N)N)C(=O)NCC(=O)N[C@@H](CC(=O)O)C(=O)N2]

A most preferred such RGD peptide is when the BTM is a peptide of formula (A):

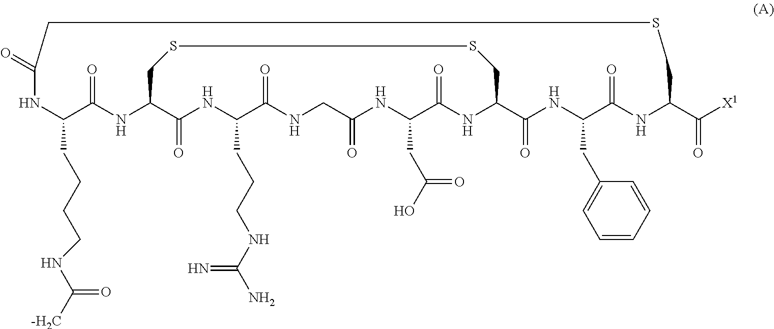

wherein X¹ is either —NH₂ or

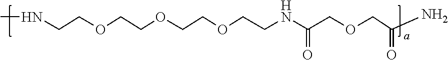

wherein a is an integer of from 1 to 10.

In Formula A, a is preferably 1.

When the BTM is a peptide, one or both termini of the peptide, preferably both, have conjugated thereto a metabolism inhibiting group (M^{IG}). Having both peptide termini protected in this way is important for in vivo imaging applications, since otherwise rapid metabolism would be expected with consequent loss of selective binding affinity for the BTM peptide. By the term “metabolism inhibiting group” (M^{IG}) is meant a biocompatible group which inhibits or suppresses enzyme, especially peptidase such as carboxypeptidase, metabolism of the BTM peptide at either the amino terminus or carboxy terminus. Such groups are particularly important for in vivo applications, and are well known to those skilled in the art and are suitably chosen from, for the peptide amine terminus:

N-acylated groups —NH(C═O)R^{G} where the acyl group —(C═O)R^{G} has R^{G} chosen from: C₁₋₆ alkyl, C₃₋₁₀ aryl groups or comprises a polyethyleneglycol (PEG) building block. Suitable PEG groups are described for the linker group (L¹), below. Preferred such PEG groups are the biomodifiers of Formulae Bio1 or Bio2 (below). Preferred such amino terminus M^{IG} groups are acetyl, benzyloxycarbonyl or trifluoroacetyl, most preferably acetyl.

Suitable metabolism inhibiting groups for the peptide carboxyl terminus include: carboxamide, tert-butyl ester, benzyl ester, cyclohexyl ester, amino alcohol or a polyethyleneglycol (PEG) building block. A suitable M^{IG} group for the carboxy terminal amino acid residue of the BTM peptide is where the terminal amine of the amino acid residue is N-alkylated with a C₁₋₄ alkyl group, preferably a methyl group. Preferred such M^{IG} groups are carboxamide or PEG, most preferred such groups are carboxamide.

When the BTM is an enzyme inhibitor, it is preferably a caspase-3 inhibitor. Such inhibitors are known in the art [Smith et al, Anti-Cancer Agents in Medicinal Chemistry, 9, 958-967 (2009)].

A preferred caspase-3 inhibitor is the isatin derivative of Formula A,

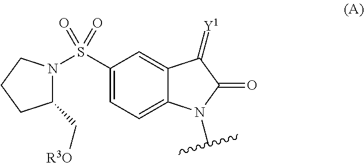

wherein: R³ comprises a group chosen from: phenyl, 3-fluorophenyl, 2,4-difluorophenyl, 3,5-difluorophenyl, tetrahydropyran, diazine or triazole;
Y¹ is O or O^{PGP}, where O^{PGP} is a protected ketone group.

When the BTM is an isatin of Formula (A), L¹ is preferably CH₂, such that the compound of Formula (I) is of Formula IA:

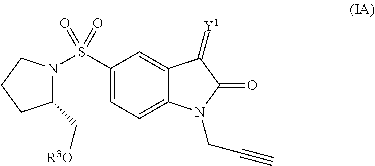

Wherein Y¹ and R³ are as defined for Formula (A).:

In Formula (A), R³ is preferably 2,4-difluorophenyl i.e. the isatin derivative is more preferably of Formula B,

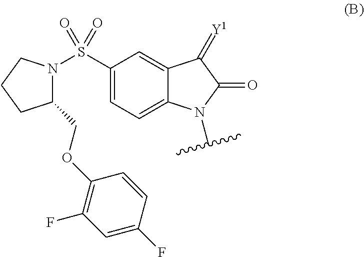

such that the compound of Formula (I) is of Formula (IB):

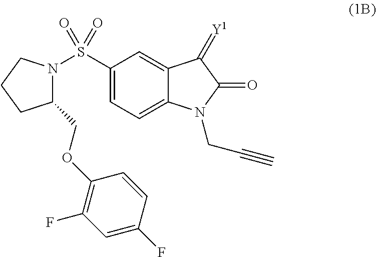

In Formulae A, B, IA and IB, Y¹ is preferably O^{PGP}. A preferred such protecting group is an acetal wherein Y¹ is —O(CH₂)_{f}O—, where f is 2 or 3. f is preferably 3. In that embodiment, the method of the first aspect preferably further comprises deprotection of the protected compound of Formula (IVA) to give the radiofluorinated product of Formula (IVB):

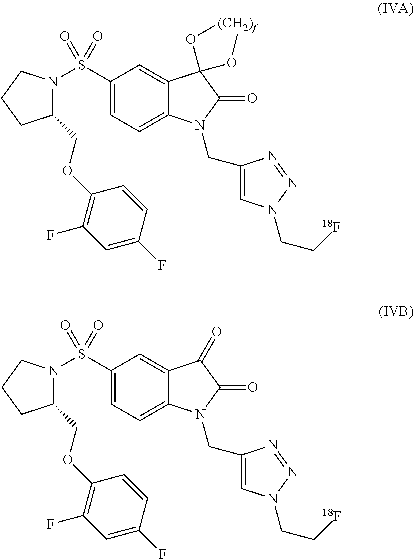

In the method of the first aspect, the linker group L¹ is preferably present. When L¹ comprises a peptide chain of 1 to 10 amino acid residues, the amino acid residues are preferably chosen from glycine, lysine, arginine, aspartic acid, glutamic acid or serine. When L¹ comprises a PEG moiety, it preferably comprises units derived from oligomerisation of the monodisperse PEG-like structures of Formulae Bio1 or Bio2:

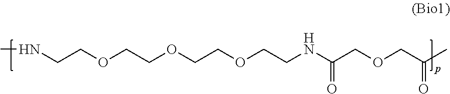

17-amino-5-oxo-6-aza-3,9,12,15-tetraoxaheptadecanoic acid of Formula Bio1 wherein p is an integer from 1 to 10. Alternatively, a PEG-like structure based on a propionic acid derivative of Formula Bio2 can be used:

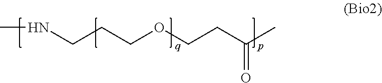

where p is as defined for Formula Bio1 and q is an integer from 3 to 15.

In Formula Bio2, p is preferably 1 or 2, and q is preferably 5 to 12.

When the linker group does not comprise PEG or a peptide chain, preferred L′ groups have a backbone chain of linked atoms which make up the -(A)ₘ- moiety of 2 to 10 atoms, most preferably 2 to 5 atoms, with 2 or 3 atoms being especially preferred. BTM peptides which are not commercially available can be synthesised by solid phase peptide synthesis as described in P. Lloyd-Williams, F. Albericio and E. Girald; Chemical Approaches to the Synthesis of Peptides and Proteins, CRC Press, 1997.

The click reaction of step (II) of the first aspect may be effected in a suitable solvent, for example acetonitrile, a C₁₋₄ alkylalcohol, dimethylformamide, tetrahydrofuran, or dimethylsulfoxide, or aqueous mixtures of any thereof, or in water. Aqueous buffers can be used in the pH range of 4-8, more preferably 5-7. The reaction temperature is preferably 5 to 100° C., more preferably at 75 to 85° C., most preferably at ambient temperature (typically 15-37° C.). The click reaction may optionally be carried out in the presence of an organic base, as is described by Meldal and Tornoe [Chem. Rev. 108, 2952, Table 1 (2008)].

The compound of Formula (I), wherein the BTM is a peptide or protein may be prepared by standard methods of peptide synthesis, for example, solid-phase peptide synthesis, for example, as described in Atherton, E. and Sheppard, R. C.; Solid Phase Synthesis; IRL Press: Oxford, 1989. Incorporation of the alkyne group in a compound of Formula (I) may be achieved by reaction of the Nor C-terminus of the peptide or with some other functional group contained within the peptide sequence, modification of which does not affect the binding characteristics of the vector. The alkyne group is preferably introduced by formation of a stable amide bond, for example formed by reaction of a peptide amine function with an activated acid or alternatively reaction of a peptide acid function with an amine function and introduced either during or following the peptide synthesis. Methods for incorporation of an alkyne group into vectors such as cells, viruses, bacteria may be found in H. C. Kolb and K. B. Sharpless, Drug Discovery Today, Vol 8 (24), 1128 December 2003 and the references therein. Alkyne derivatives are described by Glaser and Arstad [Bioconj. Chem., 18, 989-993 (2007)]. The same authors also describe methods of introducing alkyne groups into peptides.

The alkyne-functionalised isatin of Formula (IB) can be prepared by the method of Glaser [Biorg. Med. Chem. Lett, 21, 6945-6949 (2011)]. Smith et at provide the syntheses of alkyne-functionalised isatin precursors, where the isatin compound is specific for caspase-3 and caspase-7 [J. Med. Chem., 51(24), 8057-8067 (2008)]. Further approaches to functionalising BTMs with alkyne groups are described by Nwe et at [Cancer Biother. Radiopharm., 24(3), 289-302 (2009)] and Glaser et al, [J. Lab. Comp. Radiopharm., 52, 407-414 (2009)]. De Graaf et at [Bioconj. Chem., 20(7), 1281-1295 (2009)] describe non-natural amino acids having alkyne side chains and their site-specific incorporation in peptides or proteins for subsequent click conjugation. Example 4 (below) provides a bifunctional alkyne-maleimide, which can be used to conjugate with the thiol group of a thiol-containing BTM to introduce an alkyne group suitable for subsequent click reaction.

The azides of Formula (II) can be obtained as described by Demko and Sharpless, by conversion of the corresponding bromo-alcohol of formula Br—(CH₂)ₙ—OH to the corresponding azido-alcohol N₃—(CH₂)ₙ—OH, followed by conversion to the tosylate with toluenesulfonyl chloride in the presence of triethylamine [Org. Lett., 3(25), 4091-4094 (2001)]. An alternative method is S_{N}2 displacement with azide of a ditosylate species as detailed below (reaction 1). A further method is described for PEGylated chains in Svedhem et al, J. Org. Chem., 2001, p 4494 (reaction 2):

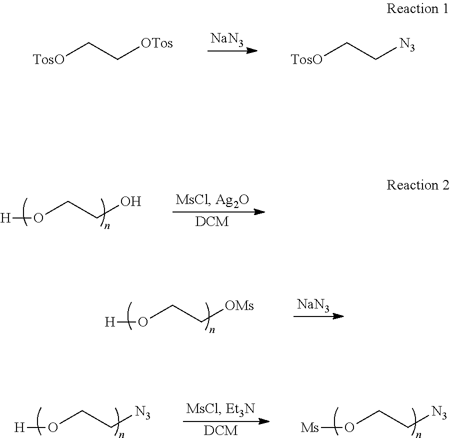

where: DCM=dichloromethane, MsCl=methanesulfonyl chloride.

The synthesis of the N₃—(CH₂)ₙ—OSO₂R¹ azide of Formula (II) using the method of Demko is preferred because of the facile protocol and ease of purification.

The method of the first aspect is preferably carried out in an aseptic manner, such that the radiofluorinated product of Formula (IV) is obtained as a radiopharmaceutical composition. The radiopharmaceutical composition comprises an effective amount of a compound of Formula (IV), together with a biocompatible carrier medium.

The “biocompatible carrier medium” comprises one or more pharmaceutically acceptable adjuvants, excipients or diluents. It is preferably a fluid, especially a liquid, in which the compound of Formula (IV) is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

When the product is a radiopharmaceutical composition, the method of the first aspect is carried out under aseptic manufacture conditions to give the desired sterile, non-pyrogenic radiopharmaceutical product. It is preferred therefore that the key components, especially any parts of the apparatus which come into contact with the product of Formula (IV), (eg. vials and transfer tubing) are sterile. The components and reagents can be sterilised by methods known in the art, including: sterile filtration, terminal sterilisation using e.g. gamma-irradiation, autoclaving, dry heat or chemical treatment (e.g. with ethylene oxide). It is preferred to sterilise the non-radioactive components in advance, so that the minimum number of manipulations need to be carried out on the radiopharmaceutical product. As a precaution, however, it is preferred to include at least a final sterile filtration step.

The compounds of Formulae (I) and (II) or (III), plus optional click catalyst and other such reagents and solvents are each supplied in suitable vials or vessels which comprise a sealed container which permits maintenance of sterile integrity and/or radioactive safety, plus optionally an inert headspace gas (eg. nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (eg. to change the headspace gas or degas solutions), and withstand pressure changes such as reductions in pressure without permitting ingress of external atmospheric gases, such as oxygen or water vapour. The reaction vessel is suitably chosen from such containers, and preferred embodiments thereof. The reaction vessel is preferably made of a biocompatible plastic (eg. PEEK).

The radiopharmaceutical composition method of the first aspect is preferably carried out using an automated synthesizer apparatus. By the term “automated synthesizer” is meant an automated module based on the principle of unit operations as described by Satyamurthy et at [Clin. Positr. Imag., 2(5), 233-253 (1999)]. The term ‘unit operations’ means that complex processes are reduced to a series of simple operations or reactions, which can be applied to a range of materials. Such automated synthesizers are preferred for the method of the present invention especially when a radiopharmaceutical product is desired. They are commercially available from a range of suppliers [Satyamurthy et al, above], including: GE Healthcare; CTI Inc; Ion Beam Applications S. A. (Chemin du Cyclotron 3, B-1348 Louvain-La-Neuve, Belgium); Raytest (Germany) and Bioscan (USA).

Commercial automated synthesizers also provide suitable containers for the liquid radioactive waste generated as a result of the radiopharmaceutical preparation. Automated synthesizers are not typically provided with radiation shielding, since they are designed to be employed in a suitably configured radioactive work cell. The radioactive work cell provides suitable radiation shielding to protect the operator from potential radiation dose, as well as ventilation to remove chemical and/or radioactive vapours.

Preferred automated synthesizers of the present invention are those which comprise a disposable or single use cassette which comprises all the reagents, reaction vessels and apparatus necessary to carry out the preparation of a given batch of radiopharmaceutical. Such cassettes are described in the fifth aspect (below). The cassette means that the automated synthesizer has the flexibility to be capable of making a variety of different radiopharmaceuticals with minimal risk of cross-contamination, by simply changing the cassette. The cassette approach also has the advantages of: simplified set-up hence reduced risk of operator error; improved GMP (Good Manufacturing Practice) compliance; multi-tracer capability; rapid change between production runs; pre-run automated diagnostic checking of the cassette and reagents; automated barcode cross-check of chemical reagents vs the synthesis to be carried out; reagent traceability; single-use and hence no risk of cross-contamination, tamper and abuse resistance.

In a second aspect, the present invention provides a method of preparation of a conjugate of Formula (III):

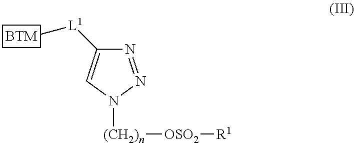

which comprises click reaction of a compound of Formula (I) with an azide of Formula (II):

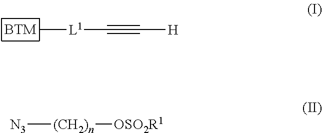

wherein: BTM, L¹, n and R¹ are as defined in the first aspect (above).

Preferred embodiments of BTM, L¹, n and R¹ in the second aspect are as defined in the first aspect (above).

The conjugate of Formula (III) could alternatively be prepared via click reaction of an azido-alcohol N₃—(CH₂)ₙ—OH, followed by formation of the sulfonate ester. That route has several disadvantages. First, the sulfonate ester formation must be carried out in the presence of the BTM, which risks side-reactions and possible compromise or loss of activity of the BTM. Secondly, the azido-alcohol (n=1-4) is a small molecule species that is potentially explosive. Thirdly, such azido-alcohol species lack a chromophore and are thus more difficult to visualise by common organic chemistry laboratory techniques such as TLC. That has a detrimental effect on product purification.

In a third aspect, the present invention provides the use of the conjugate of Formula (III) as defined in the first aspect, in the radiofluorination method of the first aspect.

Preferred embodiments of the conjugate of Formula (III) in the third aspect are as defined in the first aspect (above).

In a fourth aspect, the present invention provides the use of the azide of Formula (II) as defined in the first aspect in the radiofluorination method of the first aspect, or the method of preparation of the second aspect. Preferred embodiments of the azide of Formula (II) in the fourth aspect are as defined in the first aspect (above).

In a fifth aspect, the present invention provides a single use, sterile cassette suitable for use in the preferred automated synthesizer radiopharmaceutical composition preparation method of the first aspect, said cassette comprising either:
(i) separate supplies of the compound of Formula (I) and the azide of Formula (II) as defined in the first aspect; or
(ii) the conjugate of Formula (III) as defined in the first aspect.

Preferred embodiments of the compound of Formula (I), the azide of Formula (II) and the conjugate of Formula (III) in the fifth aspect are as defined in the first aspect (above). In the fifth aspect, preferably the BTM of the conjugate of Formula (III) does not comprise an isatin derivative of Formula (A) or (B) as defined in the first aspect.

By the term “cassette” is meant a piece of apparatus designed to fit removably and interchangeably onto an automated synthesizer apparatus (as defined above), in such a way that mechanical movement of moving parts of the synthesizer controls the operation of the cassette from outside the cassette, i.e. externally. Suitable cassettes comprise a linear array of valves, each linked to a port where reagents or vials can be attached, by either needle puncture of an inverted septum-sealed vial, or by gas-tight, marrying joints. Each valve has a male-female joint which interfaces with a corresponding moving arm of the automated synthesizer. External rotation of the arm thus controls the opening or closing of the valve when the cassette is attached to the automated synthesizer. Additional moving parts of the automated synthesizer are designed to clip onto syringe plunger tips, and thus raise or depress syringe barrels.

The cassette is versatile, typically having several positions where reagents can be attached, and several suitable for attachment of syringe vials of reagents or chromatography cartridges (eg. SPE). The cassette always comprises a reaction vessel. Such reaction vessels are preferably 1 to 10 cm³, most preferably 2 to 5 cm³ in volume and are configured such that 3 or more ports of the cassette are connected thereto, to permit transfer of reagents or solvents from various ports on the cassette. Preferably the cassette has 15 to 40 valves in a linear array, most preferably 20 to 30, with 25 being especially preferred. The valves of the cassette are preferably each identical, and most preferably are 3-way valves. The cassettes of the present invention are designed to be suitable for radiopharmaceutical manufacture and are therefore manufactured from materials which are of pharmaceutical grade and ideally also are resistant to radiolysis.

In a sixth aspect, the present invention provides the use of an automated synthesizer apparatus to carry out the radiofluorination method of the first aspect. Preferred embodiments of the automated synthesizer, and radiofluorination method in the sixth aspect are as described in the first aspect (above). The automated synthesizer of the seventh aspect preferably comprises a cassette as described in the sixth aspect (above).

The invention is illustrated by the following Examples. Example 1 provides the synthesis of Compound 2 of the invention, via click cyclisation of a tosyl-azide derivative with an alkyne-functionalised isatin. Example 2 provides a cassette configuration or the automated synthesis of Compound 4 using a FastLab™ automated synthesizer. Example 3 provides the automated synthesis of Compound 4 of the invention. Example 4 provides the synthesis of a bifunctional alkyne-maleimide, suitable for covalent conjugation with the thiol groups of a BTM to introduce alkyne groups.

### ABBREVIATIONS

BPDS: disodium 4,4′-(1,10-phenanthroline-4,7-diyl)dibenzenesulfonate,

DCM: dichloromethane,

DIEA: diisopropylethylamine,

DMF: dimethylformamide,

HPLC: high performance liquid chromatography

MeCN: acetonitrile

PAA: peracetic acid,

RCP: radiochemical purity

RT: room temperature.

t_{R}: retention time.

### Compounds of the Invention.

| Com- | |
|---|---|
| pound | Formula |
| 1 | 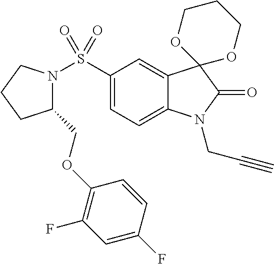 [SMILES: C#CCN1C(=O)C2(OCCCO2)c2cc(S(=O)(=O)N3CCC[C@H]3COc3ccc(F)cc3F)ccc21] |
| 2 | 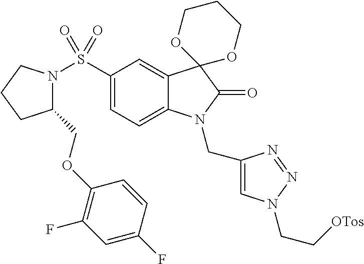 |
| 3 | 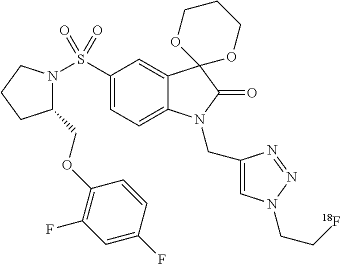 [SMILES: O=C1N(Cc2cn(CC[18F])nn2)c2ccc(S(=O)(=O)N3CCC[C@H]3COc3ccc(F)cc3F)cc2C12OCCCO2] |
| 4 | 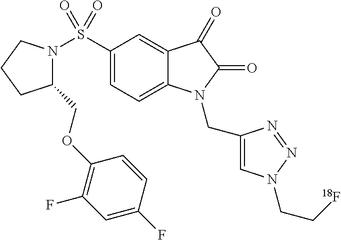 [SMILES: O=C1C(=O)N(Cc2cn(CC[18F])nn2)c2ccc(S(=O)(=O)N3CCC[C@H]3COc3ccc(F)cc3F)cc21] |

Tos = tosylate group.

### Example 1

### Synthesis of Compound 2

Compound 1 was obtained by the method of Glaser [Biorg. Med. Chem. Lett, 21, 6945-6949 (2011)] and Smith [J. Med. Chem., 51, 8057-8067 (2008)]. Toluene-4-sulfonic acid-2-azidoethyl ester was obtained by the method of Demko and Sharpless [Org. Lett., 3(25), 4091-4094 (2001)].

To a stirred solution of Compound 1 (52 mg, 0.1 mmol) in DMF (2 mL) was added copper sulfate (13 mg, 0.05 mmol) in water (0.2 mL) followed by ascorbic acid (18 mg, 0.1 mmol) in water (0.2 mL) and then toluene-4-sulfonic acid-2-azidoethyl ester (29 mg, 0.12 mmol) in dry DMF (0.5 mL) and the mixture left to stir under argon. After 4 h, TLC indicated reaction completion and mixture was poured onto water (10 mL) and extracted with DCM (3×10 mL) and dried over Na₂SO₄. Chromatography (4:1 ethyl acetate/hexanes) afforded 1 as the second fraction (first fraction is unreacted toluene-4-sulfonic acid-2-azidoethyl ester), a colourless oil that became a white foam on removal of further residual solvent (61 mg, 80%).

¹H NMR (400 MHz, CDCl₃): δ 7.88 (d, J=1.8 Hz, 1H), 7.81 (dd, J=8.2 Hz, 1.8 Hz, 1H), 7.65 (d, J=8.6 Hz, 2H), 7.62 (s, 1H), 7.28 (d, J=8.6 Hz, 2H), 7.21 (d, J=8.2 Hz, 1H), 7.03-6.96 (m, 1H), 6.88-6.77 (m, 2H), 4.99-4.96 (m, 2H), 4.92 (s, 2H), 4.60 (t, J=5.2 Hz, 2H), 4.36 (t, J=5.2 Hz, 2H), 4.30-4.26 (m, 1H), 4.02-3.88 (m, 4H), 3.53-3.47 (m, 1H), 3.10-3.03 (m, 1H), 2.43 (s, 3H), 2.41-2.37 (m, 1H), 2.06-1.93 (m, 2H), 1.75-1.63 (m, 3H).

### Example 2

### Cassette Configuration for Automated Synthesis of Compound 4

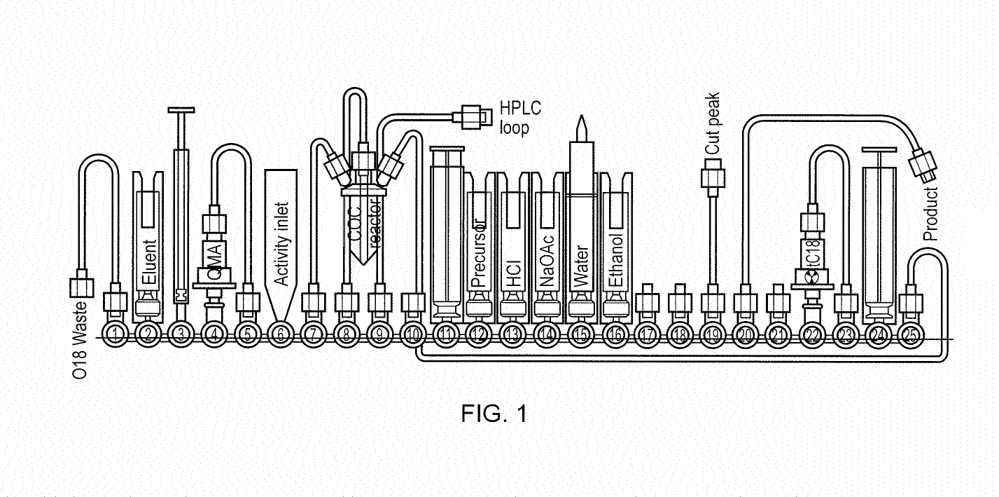

The reagents for the radiosynthesis were contained in small sealed vials or in sealed bottles as depicted in FIG. 1. Reagents were prepared and positioned as described in Table 1. These reagents were inserted into a standard [¹⁸F] FASTlab™ synthesis manifold (GE Healthcare Limited) and connected via silicone tubing. The tC18 Sep-Pak cartridge (Waters) was pre-conditioned with 2 mL of 1:1 ethanol:water followed by 10 mL of water and dried with 10 mL of air.

TABLE 1
FASTlab ™ cassette reagent positions for Compound 4 radiosynthesis.

| FASTlab manifold | |
|---|---|
| position | Reagent |
| 1 | ¹⁸O water collection |
| 2 | 1 mL 7.5 mg Kryptofix 2.2.2, 7 mg potassium |
| | hydrogen carbonate |
| 3 | 1 mL syringe |
| 4/5 | QMA-carb Sep-Pak |
| 6 | ¹⁸F inlet |
| 7/8 | Reaction vessel |
| 9 | Outlet to HPLC loop |
| 10 | Unused |
| 11 | 5 mL syringe |
| 12 | 1.4 mL Compound 2 solution |
| 13 | 4 mL 4N HCl |
| 14 | 4 mL 3N NaOAc |
| 15 | 100 mL Water bag |
| 16 | 4 mL 1:1 Ethanol:Water |
| 17/18 | Unused |
| 19 | HPLC purified product inlet |
| 20 | Product outlet |
| 21 | Unused |
| 22/23 | tC18 Sep-Pak |
| 24 | 5 mL Syringe |
| 25 | Reaction vessel |

### Example 3

### Automated Synthesis of Compound 4

No-carrier-added aqueous [¹⁸F]fluoride solution (1.5 mL, 40 GBq to 56 GBq) in enriched ¹⁸O water was delivered from the cyclotron directly to the FastLab™ synthesizer through a Teflon line by helium overpressure of the target. The activity was trapped on a Waters QMA-carbonate Sep-Pak SPE cartridge and the [¹⁸O]H₂O captured in a separate vial allowing for later recovery. 700 μL of the eluent solution (7.5 mg Kryptofix 2.2.2, 7 mg potassium hydrogen carbonate, 560 μL acetonitrile, 140 μL H₂O) was taken by syringe 1 and used to elute the activity into the COC reactor. The [¹⁸F]fluoride solution was evaporated to dryness by a combination of vacuum (−1000 mbar) and nitrogen flow (1200 mbar) at a temperature of 120° C. over an 8 minute period, resulting in a fluoride/Kryptofix 2.2.2/carbonate mixture containing 250 to 375 ppm of water, as determined by Karl Fisher titration.

Following evaporation, Compound 2 (2.85 mg; 3.75 μmol) in 1 mL of anhydrous acetonitrile was added into the reactor through its central tubing connection and the labelling reaction was conducted at 110° C. for 12.5 min in the sealed reaction vessel resulting in formation of Compound 3 in 78±3% yield (analytical). Removal of the acetal protecting group was achieved quantitatively through the addition of 1.2 mL of 4 N HCl and heating at 110° C. for 15 minutes. Once cooled to 70° C., the reaction solution was neutralised via the addition of 1.8 mL of 3N sodium acetate.

Compound 4 was purified using a Phenomenex Ultracarb ODS (30) 250×10 mm (7 μm) HPLC column with an isocratic mobile phase of 0.05M ammonium acetate and ethanol (58:42 v/v) at a flow rate of 5 mL/min. Sample injection, product isolation and data collection was performed using an in-house Multi-stream HPLC system and bespoke software package (Hammersmith Imanet Ltd., UK).

Following preparative HPLC purification, the isolated product was transferred to a 100 mL bottle of water incorporated into the FASTlab cassette resulting in a 10 fold dilution. Following homogenisation by a stream of nitrogen, the diluted product was trapped on a tC18 Sep-Pak cartridge (Waters). The cartridge was dried in a stream of nitrogen and the product eluted with 2 mL of a 1:1 ethanol:water mixture into a sterile product collect vial containing 10 mL of 0.9% injectable saline. After headspace GC residual solvent analysis, no other solvents were detected other than ethanol (8-9.2% w/v).

### Example 4

### Synthesis of Maleimide-Alkyne Bifunctional Linker (5)

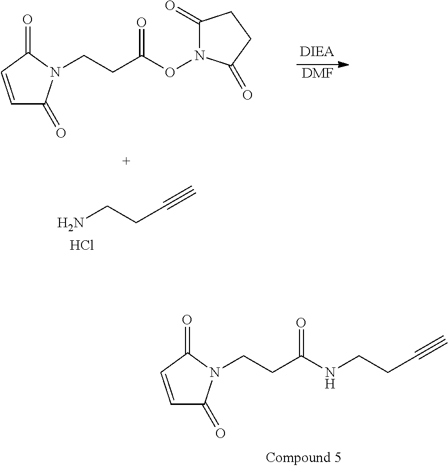

N-[β-Maleimidopropyloxy]succinimide ester (50 mg, 1.25 equiv) was dissolved in 1.0 mL of dry DMF. 3-Butyn-1-amine hydrochloride (16 mg, 1.0 equiv) was dissolved in 0.5 mL of dry DMF and 26 μL of DIEA. This amine solution was added dropwise to the succinimide ester while keeping the ester solution in an ice bath. The mixture was stirred at 0° C. for 10 min. The solution was warmed up to room temperature and stirred for 18 h. The solvents were evaporated in vacuo and the residue was dissolved in 5 mL CH₂Cl₂. The organic solution was extracted with brine (3×5 mL) and dried over MgSO₄. The solvent was removed under reduced pressure and the crude material was purified using flash chromatography (silica, MeOH/CH₂Cl₂). The product (5) was purified from grease by dissolving the sample in a minimum amount of CH₂Cl₂ (ca. 2 mL), followed by three washes with hexanes. The product (5) precipitated as a fluffy white solid. Characterization of the product was achieved using ¹H-NMR. Yield: 8.2 mg (25%).

¹H-NMR (500 MHz, CDCl₃): δ 2.02 (s, 1H), 2.41 (t, J=5 Hz, 2H), 2.57 (t, J=5 Hz, 2H), 3.42 (dt, J=5 Hz, 2H), 3.88 (t, J=5 Hz), 5.90 (bs, 1H), 6.73 (s, 2H).

What is claimed is:

1. A method of ¹⁸F-radiofluorination of a biological targeting moiety, which comprises click reaction of a compound of Formula (I) with an azide of Formula (II): 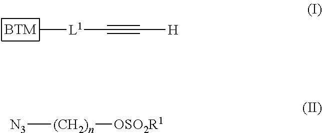
to give a conjugate of Formula (III): 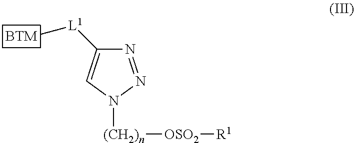
and reaction of the conjugate of Formula (III) with [¹⁸F]-fluoride to give the radiofluorinated product of Formula (IV): 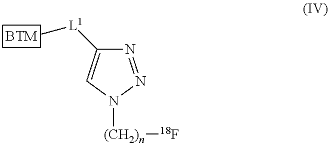
wherein:
L¹ is a linker group which may be present or absent;
n is 2, 3 or 4;
R¹ is C₁₋₄ alkyl; C₁₋₄ fluoroalkyl; or —C₆H₄—R²,
where R² is chosen from: H, CH₃, Br or NO₂;
BTM is the biological targeting moiety, optionally protected with one or more protecting groups.

2. The method of claim 1, where L¹ is a group of formula -(A)ₘ- wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, a sugar or a monodisperse polyethyleneglycol (PEG) building block;
and wherein each R is independently chosen from: H, C₁₋₄ alkyl, C₂₋₄ alkenyl, C₂₋₄ alkynyl, C₁₋₄ alkoxyalkyl or C₁₋₄ hydroxyalkyl; and
m is an integer of value 1 to 20.

3. The method of claim 1, wherein the click reaction of step (ii) is carried out in the presence of a click catalyst which comprises copper.

4. The method of claim 1, wherein the compound of Formula (I) has one or more functional groups of the BTM protected with one or more protecting group(s), and said protecting group(s) are removed after step (iii).

5. The method of claim 1, where BTM is a single amino acid, a 3-80 mer peptide, an enzyme substrate, an enzyme antagonist, an enzyme agonist, an enzyme inhibitor or a receptor-binding compound.

6. The method of claim 5, wherein BTM is an RGD peptide.

7. The method of claim 5, wherein BTM is a caspase-3 inhibitor.

8. The method of claim 7, where the caspase-3 inhibitor is an isatin derivative of Formula A, 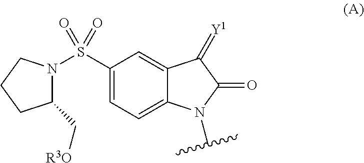
and L¹ is CH₂, such that the compound of Formula (I) is of Formula IA: 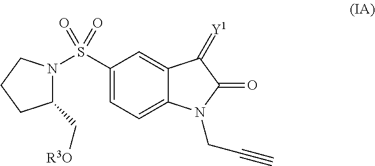
wherein:
R³ comprises a group chosen from: phenyl, 3-fluorophenyl, 2,4-difluorophenyl, 3,5-difluorophenyl, tetrahydropyran, diazine or triazole;
Y¹ is O or O^{PGP}, where O^{PGP} is a protected ketone group.

9. The method of claim 8, where the isatin derivative is of Formula B, 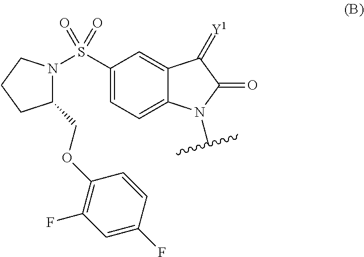
such that the compound of Formula (I) is of Formula (IB):

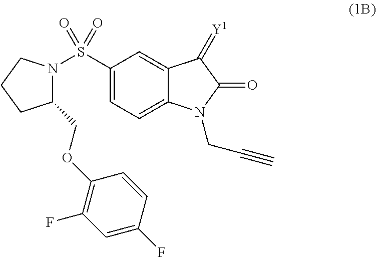

10. The method of claim 9, where Y¹ is —O(CH₂)_{f}O—, where f is 2 or 3, and the method further comprises deprotection of the protected compound of Formula (IVA) to give the radiofluorinated product of Formula (IVB):

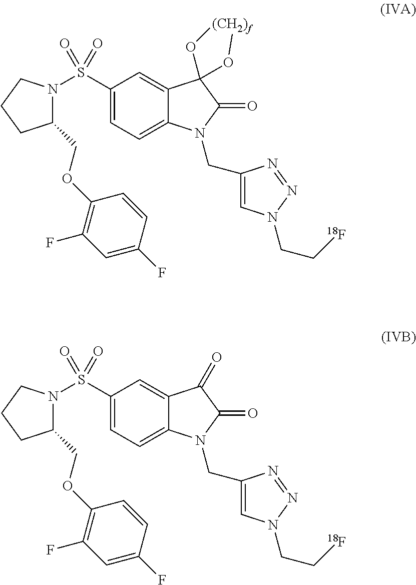

11. The method of claim 1, which is carried out in an aseptic manner, such that the radiofluorinated product of Formula (IV), (IVA) or (IVB) is obtained as a radiopharmaceutical composition.

12. The method of claim 11, which is carried out using an automated synthesizer apparatus.

13. A method of preparation of a conjugate of Formula (III): 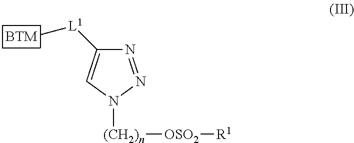
which comprises click reaction of a compound of Formula (I) with an azide of Formula (II): 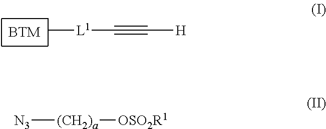
wherein: BTM, L¹, n and R¹ are as defined in claim 1.

14. (canceled)

15. (canceled)

16. A single use, sterile cassette suitable for use in the automated synthesizer method of claim 11, said cassette comprising either:
(i) separate supplies of the compound of Formula (I) and the azide of Formula (II); or
(ii) the conjugate of Formula (III).

17. (canceled)

18. (canceled)
